SEQUENCE LISTING (5 sequences)

SEQ ID NO: 1 - DNA, 3219 bp, Daucus carota
  CDS (1)..(3063)
atgggtgtgttgagagtgtatgtgatcttgattcttgttgggttttgtgtgcaaattgttgtggtgaattcccagaacttgacatgtaattccaatgatttgaaggcattggaggggttcatgagaggtttagaatcaagtattgatgggtggaaatggaatgaaagttcatctttttcatcaaattgttgtgattgggtaggcataagttgcaagtcttctgtttctcttggactagatgatgtaaacgagtctggtagggtagtagagttggagcttgggaggagaaaattgagtggcaagctttcggaatcagtagccaagttagatcagctaaaggttcttaatttaactcacaattcattgagtggctctatagctgcatcactgctgaatttgagcaatttagaggttttggacttgagcagcaatgacttttctggattgtttccaagtttgatcaacttaccttcgcttcgagttttgaacgtatatgaaaattcttttcatggtctcatacctgctagtttgtgcaacaatttgccccgtattagagagattgatttggcaatgaattattttgatgggagtattccggtggggattggaaattgcagctcagtggagtatcttggtcttgcttcaaacaatctatccggcagtattccgcaggagttgtttcagttatcaaatttgtctgtattggctcttcagaacaacaggctctctggggcattgagcagcaaacttggtaaactttccaaccttggtcgtttggatatttcttcaaataaattttcagggaagataccagatgtttttcttgagttgaacaaattatggtatttttcagctcaatcaaatcttttcaatggtgaaatgcctaggtcattgtcgaattctcggtctatttctttgcttagtttgaggaacaatacattaagtggtcagatttatcttaattgctctgcaatgactaatcttacatcacttgatctggcttccaattccttcagtggatccatcccatctaatttacccaactgtctgagattgaaaaccataaattttgctaaaatcaaattcatcgctcaaatcccagaaagtttcaagaattttcagagtctgacttctctttctttctcaaattctagtattcaaaacatttcatctgccctagaaattttacagcattgccagaacttaaaaactttggtgcttaccttgaattttcagaaagaagaattaccatctgttcccagtctgcagttcaaaaaccttaaggttttaataattgccagttgccaacttaggggtaccgttccgcagtggctgagtaattctccatcattgcagttgttggatttgtcttggaatcagttgagtggaacaattccaccttggttaggcagcttgaattccctcttttacctcgatttatcgaacaacacgtttatcggtgagattccgcatagcctcaccagtttacagagccttgtctccaaggagaacgctgtagaagagccctcaccagattttccatttttcaagaaaaaaaacacaaatgccggagggttgcagtataatcagccttcgagcttcccacctatgatagaccttagttataattccctcaatgggtcaatctggccagaatttggggatctgcggcagctgcacgttttgaacctgaaaaacaataatttgtcaggaaacattccagccaacttgtcaggtatgactagcttggaagtcttggatttgtcccataacaatctctcgggtaatatacctccttccctggtgaaacttagctttttgtcaacgtttagcgttgcatacaataagctatcgggcccaattcccacaggtgtccaatttcaaacctttcctaactcgagtttcgaagggaaccaaggtctatgtggtgagcatgcttccccatgtcatattactgatcaatcaccccatggatcagctgtcaaatcaaagaaaaatatacgaaaaatagttgcagtggctgttgggactggtcttggaacagtttttcttctcactgttactttattgattattctgcggacaaccagccgaggagaggttgatcccgagaagaaggcagatgctgatgaaattgagcttggttcaagatcagtggtacttttccataacaaggacagtaataacgagctctcacttgatgacattttgaaatccactagcagttttaatcaagcaaacattatcggctgtgggggctttggcttggtatacaaagccacccttcctgatggtacaaaggttgcgatcaaacgactctctggtgacactggtcagatggatagagaatttcaggctgaagttgaaacgctttcaagagctcagcatccgaaccttgtccatcttctggggtattgcaattataagaatgataaactcctaatatactcatacatggataatggtagcttggattattggctgcatgagaaagtggatggacctccttcattagattggaaaaccaggcttcgtatcgctcgaggggcagcagaaggactggcttacttgcaccaatcatgtgagccccatattcttcaccgcgatataaagtctagtaatatccttctaagtgatacgtttgtagctcacttggcagattttggtcttgctagactcatacttccatatgatactcatgttaccactgacctagttggaactttggggtacattccacccgaatatggacaagcttctgtggcaacatacaagggggatgtctatagcttcggagtggttctcttagagcttcttactggtaggaggccaatggatgtgtgtaaaccaagaggaagtcgagatttaatatcctgggttctacaaatgaagacagagaaaagagagagtgaaatatttgatccctttatttatgacaaagaccatgctgaagaaatgttgttggttcttgagattgcttgccgctgcttaggtgaaaaccctaaaacaagacctacaacacaacagctagtatcttggctcgaaaacattgatgtcagtagttagcattgtcctgtcattgtttagtaaatcaaaacaattggctcattaatagatcctggcaatttgcattgctcagcttgaaatagtgtattaataagtttggtgtatagattatacatgaggaagtttctttctttcaaaaaaaaaaaaaaaaaa

SEQ ID NO: 2 - protein, 1021 aa, Daucus carota
MGVLRVYVILILVGFCVQIVVVNSQNLTCNSNDLKALEGFMRGLESSIDGWKWNESSSFSSNCCDWVGISCKSSVSLGLDDVNESGRVVELELGRRKLSGKLSESVAKLDQLKVLNLTHNSLSGSIAASLLNLSNLEVLDLSSNDFSGLFPSLINLPSLRVLNVYENSFHGLIPASLCNNLPRIREIDLAMNYFDGSIPVGIGNCSSVEYLGLASNNLSGSIPQELFQLSNLSVLALQNNRLSGALSSKLGKLSNLGRLDISSNKFSGKIPDVFLELNKLWYFSAQSNLFNGEMPRSLSNSRSISLLSLRNNTLSGQIYLNCSAMTNLTSLDLASNSFSGSIPSNLPNCLRLKTINFAKIKFIAQIPESFKNFQSLTSLSFSNSSIQNISSALEILQHCQNLKTLVLTLNFQKEELPSVPSLQFKNLKVLIIASCQLRGTVPQWLSNSPSLQLLDLSWNQLSGTIPPWLGSLNSLFYLDLSNNTFIGEIPHSLTSLQSLVSKENAVEEPSPDFPFFKKKNTNAGGLQYNQPSSFPPMIDLSYNSLNGSIWPEFGDLRQLHVLNLKNNNLSGNIPANLSGMTSLEVLDLSHNNLSGNIPPSLVKLSFLSTFSVAYNKLSGPIPTGVQFQTFPNSSFEGNQGLCGEHASPCHITDQSPHGSAVKSKKNIRKIVAVAVGTGLGTVFLLTVTLLIILRTTSRGEVDPEKKADADEIELGSRSVVLFHNKDSNNELSLDDILKSTSSFNQANIIGCGGFGLVYKATLPDGTKVAIKRLSGDTGQMDREFQAEVETLSRAQHPNLVHLLGYCNYKNDKLLIYSYMDNGSLDYWLHEKVDGPPSLDWKTRLRIARGAAEGLAYLHQSCEPHILHRDIKSSNILLSDTFVAHLADFGLARLILPYDTHVTTDLVGTLGYIPPEYGQASVATYKGDVYSFGVVLLELLTGRRPMDVCKPRGSRDLISWVLQMKTEKRESEIFDPFIYDKDHAEEMLLVLEIACRCLGENPKTRPTTQQLVSWLENIDVSS

SEQ ID NO: 3 - DNA, 20 bp, Artificial Sequence
  Synthetic DNA
ggytcytcnacngcrttytc

SEQ ID NO: 4 - DNA, 20 bp, Artificial Sequence
  Synthetic DNA
ttraaraanggraartcngg

SEQ ID NO: 5 - DNA, 20 bp, Artificial Sequence
  Synthetic DNA
gtntaygaraaytcnttyca

(19) United States
(12) Patent Application Publication
(10) Pub. No.: US 2004/0096941 A1
(43) Pub. Date: May 20, 2004

(54) Receptor for plant cell growth factor

(76) Inventors: Yoshikatsu Matsubayashi, Nagoya-shi (JP); Youji Sakagami, Nagoya-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/443,101

(22) Filed: May 22, 2003

(30) Foreign Application Priority Data
Nov. 19, 2002 (JP) 2002-335572

(51) Int. Cl. C07K 14/415; C07H 21/04; C12N 5/04

(52) U.S. Cl. 435/69.1; 435/320.1; 435/419; 530/370; 536/23.6

(57) ABSTRACT

A phytosulfokine (PSK) receptor protein selected from the groups consisting of: (a) a protein comprising an amino acid sequence of SEQ ID No: 2, and (b) a protein comprising an amino acid sequence of SEQ ID No: 2, wherein one or a few amino acids are deleted, substituted and/or added and which is capable of responding to phytosulfokine (PSK) that is a plant cell growth factor.

### CROSS-REFERENCE TO RELATED APPLICATIONS

This application is based upon and claims the benefit of priority from the prior Japanese Patent Application No. 2002-335572, filed Nov. 19, 2002, the entire contents of which are incorporated herein by reference.

### BACKGROUND OF THE INVENTION

1. Field of the Invention

The present invention relates to a gene (a sense gene) encoding a receptor for a plant cell growth factor, more specifically, a gene encoding phytosulfokine receptor (PSK receptor) for phytosulfokine (PSK). The present invention also relates to an antisense gene having a nucleotide sequence complementary to a nucleotide sequence of the sense gene. Further, the present invention relates to a recombinant vector containing either sense gene or antisense gene, a transformant having either sense gene or antisense gene, and a transgenic plant having either sense gene or antisense gene.

Throughout this specification, phytosulfokine receptor is also referred to as “PSK receptor” and phytosulfokine is also referred to as “PSK”.

2. Description of the Related Art

Since the 1990s, there have been several reports of peptide molecules which function as intercellular signal transduction substances in higher plants. That is, it has been revealed that substances which are different from conventional plant hormones such as auxin and cytokinin play an important physiological role in various aspects of the life cycle of a plant. Further, from the results of the analysis of the entire genome of Arabidopsis thaliana, it has been expected that at least 340 different receptor-kinases exist. Accordingly, the search for a ligand to be received by each receptor is now one of the most important objects in the post-genome period.

Under such circumstances, there have come to be known two types of extracellular secretory peptides which presumably regulate proliferation and differentiation of plant cells through specific receptors. One of such peptides is phytosulfokine (PSK) and the other is CLAVATA3 (CLV3) (refer to Jpn. Pat. Appln. KOKAI Publication Nos. 11-79612 and 10-45797, and A. E. Trotochaud, S. Jeong, and S. E. Clark, CLAVATA3, a multimeric ligand for the CLAVATA1 receptor-kinase., “Science”, (USA), 2000, vol. 289, pp. 613-617). Each of these peptides is translated as a precursor having a signal sequence, subjected to the subsequent processing and then secreted to the extracellular region.

Also, it has been generally known that, even after plant cells have differentiated to cells having a specific function, such differentiated cells can dedifferentiate and then redifferentiate in each appropriate condition, thereby eventually regenerating a complete plant. By utilizing this nature (what is called totipotency) which is characteristic of plants, a technology that enables producing clone plants having genes identical with the parent plant has already been established in a number of plant species. This technique enables mass-production of clones in a plant variety having higher added value, and therefore this technique now takes an important place in industrial terms. In order to induce fully such a potential capacity possessed by the plant cells, it is generally required that plant tissues are taken out under axenic conditions and they are cultured in a medium containing auxin and cytokinin as plant hormones in addition to inorganic salts, vitamins and organic components such as sugars. Further, it is possible to determine the directionality of redifferentiation of the culture tissue, i.e., to determine to which of shoot and root the culture tissue preferentially differentiates, by changing the concentration and/or the ratio of these two types of plant hormones.

As described above, nowadays, the process of proliferation and differentiation of plant cells can be, for the most part, artificially regulated. However, the dependency of cell growth on cell density, which is one of the problems which have puzzled researchers for a long time, still remains unsolved. This is the phenomenon that, plant cells, which are capable of vigorously dividing and growing when the cells are cultured as cell population such as tissue culture, exhibit extremely suppressed growth when the cells are separated from each other by an enzymatic or mechanical method and cultured as a single cell, generally when the cell density is decreased to about 10⁴ cells/ml or less. Due to this phenomenon, if the amount of the target cells is small, as is in a gene-introducing experiment, a technique called “nurse culture”, which enhances the cell density as a whole by using appropriate cells (nurse cells), is sometimes employed.

With regard to the question of why plant cells are incapable of growing at a low cell density, there has been proposed the following model on the basis of several experimental results. The model is based on the concept that plant cells which have been transferred to a culture system secrete an unknown growth factor to the extracellular region, and only when the extracellular concentration of the growth factor exceeds a given value, are the plant cells capable of starting cell division. Accordingly, when the cell density is low, it takes a long time for the growth factor to reach the required concentration and the rate at which the growth factor is degraded in the medium exceeds the rate at which it is secreted to the medium, and thus cell growth is suppressed. Actually, it has been confirmed that a medium which has been used for cell culture, i.e., a conditioned medium (CM), contains a factor which accelerates cell growth, and several studies for revealing what this factor is have been attempted.

As plants do not have highly differentiated organs like animals do, and as plant hormones such as auxin and cytokinin induce a number of physiological activities in a wide range of cell growth and differentiation, only a relatively small number of researchers have ever contemplated the possibility that a peptide growth factor such as animal hormones exists in plants. However, as a result of the discovery of PSK and CLAVATA that are peptide growth factors in the late 1990s, a concept which assumes the existence of peptide growth factors in plants has been well accepted in recent years. Under such circumstances as described above, there has been a demand for progress in the analysis of a receptor for a peptide growth factor. It is expected that revealing the physiological function of the receptor by analyzing it will result in a novel discovery in the growth control mechanism of plant cells.

As described above, there has been a demand on confirming the phenomenon that cell density significantly affects growth of plant cells, establishing a bioassay system, isolating a growth factor and determining the structure thereof, cloning the gene and analyzing a receptor which specifically receives the growth factor. However, most parts of the physiological role that PSK plays in an intact plant are still unknown, and the analysis of such a function of PSK remains as one of the important objects in the art. Further, there has been no report that a pair of a ligand and a receptor thereof, which is involved with growth of plant cells (cultured cells, in particular), has been identified. Therefore, there has been a strong demand to identify the PSK receptor, in particular.

### BRIEF SUMMARY OF THE INVENTION

An object of the present invention is to provide a gene of a receptor which regulates growth of plant cells, and to provide a technique which enables controlling the growth rate of plant cells by regulating the expression of the gene.

As a result of assiduous study for solving the aforementioned problems, the inventors of the present invention have succeeded in isolating a gene encoding the PSK receptor from carrot cells, thereby completing the present invention. Specifically, the inventors of the present invention searched for a protein which specifically interacts with PSK among the solubilized proteins of carrot cells. The inventors then discovered the protein of the PSK receptor and also succeeded in cloning of the gene. Further, the inventors discovered that proliferation and differentiation of the cells are regulated by the interaction of the PSK receptor with PSK and then succeeded in controlling the growth rate of plant cells by artificially regulating the expression of the PSK receptor gene, thereby completing the present invention.

More specifically, the present invention provides one of the following protein (a) or protein (b).

(a) a protein comprising an amino acid sequence of SEQ ID No: 2.

(b) a protein comprising an amino acid sequence of SEQ ID No: 2, wherein one or a few amino acids are deleted, substituted and/or added and which is capable of responding to phytosulfokine (PSK) that is a plant cell growth factor.

Further, the present invention provides a gene encoding one of the following protein (a) or protein (b).

(a) a protein comprising an amino acid sequence of SEQ ID No: 2.

(b) a protein comprising an amino acid sequence of SEQ ID No: 2, wherein one or a few amino acids are deleted, substituted and/or added and which is capable of responding to phytosulfokine (PSK) that is a plant cell growth factor.

Further, the present invention provides one of the following gene (c), gene (d) or gene (e).

(c) a gene having a nucleotide sequence of SEQ ID No: 1.

(d) a gene having a nucleotide sequence of SEQ ID No: 1, wherein one or a few nucleotides are deleted, substituted and/or added and which encodes a protein that is capable of responding to phytosulfokine (PSK) that is a plant cell growth factor.

(e) a gene having a nucleotide sequence which can hybridize with a complementary strand of a nucleotide sequence of SEQ ID No: 1 under a stringent condition and which encodes a protein that is capable of responding to phytosulfokine (PSK) that is a plant cell growth factor.

Further, the present invention provides a nucleic acid (i.e., antisense gene) which has a nucleotide sequence complementary to any one of the aforementioned genes and whose expression in a plant cell cause the plant cell to suppress response to PSK that is a plant cell growth factor.

Yet further, the present invention provides a recombinant vector containing any one of the aforementioned genes, and a transformant and a transgenic plant containing any one of the aforementioned genes.

In addition, the present invention provides a method of preparing a plant cell whose responsive property to a plant cell growth factor PSK is enhanced, the method comprising:

(1) introducing any one of the aforementioned gene into a plant cell, thereby obtaining the transformed cell; and

(2) culturing the transformed plant cell in a medium where the cell is allowed to proliferate.

The present invention also provides a method of preparing a redifferentiated plant whose responsive property to a plant cell growth factor PSK is enhanced, the method comprising:

(1) introducing any one of the aforementioned genes into a plant cell, thereby obtaining the transformed cell;

(2) culturing the transformed plant cell in a growth medium where the cell is allowed to proliferate; and

(3) redifferentiating the proliferated cell in a redifferentiation medium where the cell is allowed to redifferentiate.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiment of the invention, and together with the general description given above and the detailed description of the preferred embodiment given below, serve to explain the principles of the invention.

FIG. 1 is a photograph of SDS-PAGE analysis based on photoaffinity labeling of the PSK-binding proteins.

FIG. 2 is a photograph of SDS-PAGE analysis of the affinity-purified proteins.

FIG. 3 is a view showing a reversed-phase HPLC profile of the tryptic digest of the purified 120-kD protein.

FIG. 4A is a view showing a nucleotide sequence of cDNA encoding the 120-kD protein, as well as the deduced amino acid sequence.

FIG. 4B is a view schematically showing the 120-kD receptor kinase.

FIG. 5 is a photograph of a northern blot analysis, which shows the 120-kD receptor mRNA.

FIGS. 6A to 6C are photographs showing growth of the sense transformants, the antisense transformants and the control cells, respectively.

FIG. 7A is a view showing specific binding of PSK to the membranes of the sense transformants and the control cells.

FIG. 7B is a view showing Scatchard plot of the binding data in FIG. 7A.

FIG. 8 is a photograph of SDS-PAGE analysis based on photoaffinity labeling of the membrane proteins derived from the sense transformants and the control cells.

FIG. 9 is a view showing inhibition of binding of PSK to the membrane fractions of the sense transformants by competitive molecules of PSK.

FIG. 10A is a photograph showing regeneration ability in the control cells.

FIG. 10B is a photograph showing loss of regeneration ability in the sense transformants.

### DETAILED DESCRIPTION OF THE INVENTION

### 1. Isolation of the Gene of the Present Invention

The cDNA encoding the PSK receptor of the present invention can be isolated according to the conventional method, as described in detail in the following examples. Nucleotide sequence of the isolated cDNA encoding the PSK receptor is shown by SEQ ID No: 1.

Hereinafter, a method of isolating the cDNA encoding the PSK receptor will be described briefly. A PSK-binding protein is affinity-purified from a microsomal membrane of carrot cells by means of a PSK-based affinity column. The affinity-purified protein (i.e., 120-kD protein) is subjected to tryptic digestion, and the resultant peptide fragments are separated and collected by a reversed-phase HPLC. An amino acid sequence of each peptide fragment corresponding to each peak is analyzed by using a protein sequencer and mass spectrometry (MALDI-TOF MS), whereby the partial amino acid sequence of the protein is obtained. Degenerate primers are designed on the basis of the amino acid sequence obtained as described above, and a PCR reaction is carried out by using cDNA library prepared from a carrot cell as a template for PCR. The resulting PCR product is used as a hybridization probe for PSK receptor gene. The cDNA library of carrot cells is subjected to screening by using the PCR product as the probe, whereby full-length cDNA encoding the PSK receptor can be obtained.

Alternatively, the cDNA encoding the PSK receptor of the present invention can be obtained by designing primers on the basis of the nucleotide sequence of SEQ ID No: 1 and employing the conventional PCR method. Further, the cDNA encoding the PSK receptor can be obtained by effecting chemical synthesis or hybridizing a cDNA library with a DNA fragment having the nucleotide sequence of SEQ ID No: 1 as a hybridization probe.

Specifically, in a case in which the PCR method is used, cDNA library as a template and PCR primers may be prepared as described below.

First, mRNA is to be prepared, when cDNA library as a template is prepared. The preparation of mRNA can be carried out according to the conventional method. For example, a plant or plant cells (such as NC cells of carrot) are subjected to ultrasonic treatment or homogenized in a mortar, and the resultant extract is treated according to the glyoxal method, the guanidine thiocyanate-cesium chloride method, the lithium chloride-urea method, the proteinase K-deoxyribonuclease method and the like, thereby preparing a coarse RNA fraction. Thereafter, poly(A)⁺RNA (i.e., mRNA) can be obtained from this coarse RNA fraction, according to the batch method or the affinity column method using poly U-Sepharose, in which oligo dT-cellulose or Sepharose2B is used as a carrier. The resultant mRNA may optionally be subjected to further purification by sucrose density-gradient centrifugation or the like. By using the mRNA obtained as described above as a template and a commercially available kit (e.g., ZAP-cDNA Synthesis Kit manufactured by STRATAGENE Co., Ltd.), single-stranded cDNA is synthesized with oligo dT₂₀ and a reverse transcriptase. Then, double-stranded cDNA is synthesized from this single-stranded cDNA. Thereafter, an appropriate adapter is added to the double-stranded cDNA obtained as descried above, and the resultant double-stranded cDNA is connected to an appropriate plasmid, whereby a cDNA library is prepared.

Regarding the PCR primer, E1, E2 and F primers (refer to the examples describe below), which have been actually used for isolating the PSK receptor-encoding cDNA of the present invention, may be used. In a case in which PSK receptor-encoding cDNA of a plant other than carrot is prepared from a cDNA library of the plant by means of PCR, degenerate primers may be designed on the basis of the nucleotide sequence of the PSK receptor-encoding cDNA.

By using the aforementioned cDNA library as a template and the aforementioned PCR primers and carrying out PCR in a condition commonly practiced, the PSK receptor-encoding cDNA can be obtained.

The obtained PSK receptor-encoding cDNA can be cleaved with a restriction enzyme and then inserted into a commercially available plasmid. The resulting recombinant plasmid is isolated and purified according to a conventional method (for example, J. Sambrook et al., Molecular Cloning, 2nd Ed., Cold Spring Harbour Laboratory Press, pp. 1.21-1.52). Further, the nucleotide sequence of the PSK receptor-encoding cDNA may be confirmed according to a conventional method such as Sanger method and Maxam-Gilbert Method or by using an automatic nucleotide sequence determining device (ABI DNA sequencer 310).

SEQ ID No: 1 represents the nucleotide sequence of the PSK receptor gene of the present invention, and SEQ ID No: 2 represents the amino acid sequence encoded by the PSK receptor gene of the present invention. The protein having the amino acid sequence is also referred to as “the PSK receptor protein”. In the present invention, it is acceptable that a plurality of amino acids (preferably one or a few amino acids) in the aforementioned amino acid sequence exhibits mutation such as deletion, substitution and addition, as long as the PSK receptor protein containing the mutation is capable of responding to phytosulfokine (PSK) that is a plant cell growth factor. In other words, it is acceptable as long as the PSK receptor protein containing the mutation is capable of conferring an enhanced responsive property to PSK on a transgenic plant in which the protein-encoding gene has been introduced.

For example, 1 to 10 (preferably 1 to 5) amino acids in the amino acid sequence of SEQ ID No: 2 may be deleted (e.g., methionine as the first amino acid in the amino acid sequence of SEQ ID No: 1 may be deleted). Alternatively, 1 to 10 (preferably 1 to 5) amino acids may be added to the amino acid sequence of SEQ ID No: 2. Or, 1 to 10 (preferably 1 to 5) amino acids in the amino acid sequence of SEQ ID No: 2 may be substituted with amino acids of other types.

In the present invention, the term “being capable of responding to PSK that is a plant cell growth factor” represents that the physiological activity is induced by the action of PSK. That is, the term “being capable of responding to PSK that is a plant cell growth factor” represents that cell division and proliferation are enhanced by the action of PSK.

“A protein which is capable of responding to PSK that is a plant cell growth factor” represents a protein which is capable of conferring an enhanced responsive property to PSK on a transgenic plant in which the protein-encoding gene has been introduced. “A gene which is capable of responding to PSK that is a plant cell growth factor” represents a gene which is capable of conferring an enhanced responsive property to PSK on a transgenic plant in which the gene has been introduced.

Further, the expression “a responsive property to the plant cell growth factor PSK is enhanced” represents that the PSK-binding capacity in a transgenic plant in which the PSK receptor-encoding gene of the present invention has been expressed is 10% or more, preferably 100 to 1000% or more increased, as compared with the PSK-binding capacity in a wild type plant of the same species. That is, the expression represents that the cell division and proliferation of the transgenic plant is significantly accelerated, as compared with those of the wild type plant of the same species. Conversely, the expression “a responsive property to the plant cell growth factor PSK is suppressed” represents that PSK-binding capacity in a transgenic plant in which a gene (antisense gene) complementary to the PSK receptor-encoding gene of the present invention has been expressed is 10% or more, preferably 100 to 1000% or more decreased, as compared with PSK-binding capacity in a wild type plant of the same species. That is, the expression represents that the cell division and proliferation of the transgenic plant is significantly poor, as compared with those of the wild type plant of the same species.

The gene of the present invention includes a gene having a nucleotide sequence which can hybridize with a complementary strand of a nucleotide sequence of SEQ ID No: 1 under a stringent condition and which encodes a protein that is capable of responding to PSK that is a plant cell growth factor. The term “a stringent condition” represents a condition in which “a specific hybrid” can be formed. For example, a stringent condition may represent a condition in which two nucleic acids having high homology, i.e., two DNA strands having homology of 90% or more, preferably 95% or more therebetween hybridize with each other and two nucleic acids having homology less than 90%, preferably less than 95% fail to hybridize with each other. More specifically, “a stringent condition” represents a condition in which the concentration of sodium is in a range of 15 to 300 mM, preferably in a range of 15 to 75 mM, the temperature is in a range of 50 to 60° C., preferably in a range of 55 to 60° C.

Further, the gene of the present invention includes a gene having a nucleotide sequence of SEQ ID No: 1, wherein a plurality of nucleotides (preferably one or a few nucleotides) is deleted, substituted and/or added and which encodes a protein that is capable of responding to PSK. That is, it is acceptable that a plurality of amino acids (preferably one or a few amino acids) in the aforementioned nucleotide sequence exhibits mutation such as deletion, substitution and addition, as long as the gene containing the mutation is capable of responding to PSK that is a plant cell growth factor. In other words, it is acceptable as long as the gene containing the mutation is capable of conferring an enhanced responsive property to PSK on a transgenic plant in which the gene has been introduced.

The nucleic acid comprising a nucleotide sequence complementary to the gene of the present invention is used, for example, in the antisense method. The nucleic acid comprising a nucleotide sequence complementary to the gene (i.e., sense gene) of the present invention is also referred to as antisense gene or antisense nucleic acid, and the antisense gene includes antisense RNA. A nucleic acid having a sequence complementary to the entire nucleotide sequence or a portion thereof of the gene of the present invention (e.g., antisense RNA) is externally administered to an organism or cells. The nucleic acid (antisense RNA) administered in such a manner forms a hybrid with mRNA in the organism or cells, thereby inhibiting the process in which the genetic information of mRNA is translated into a protein. DNA information of such antisense RNA is incorporated to an expression vector, so that the antisense RNA may be expressed inside a cell. It is not necessary for the antisense RNA to be 100% complementary to the target RNA, as long as the antisense RNA generally exhibits a sufficiently good antisense effect. It suffices that the antisense RNA can suppress expression of the PSK receptor protein of the present invention. Antisense nucleic acid has 90%, preferably 95% complementarity to the gene of the present invention. Further, in order to cause a satisfactory antisense effect, the length of a complementary antisense nucleic acid is at least 15 bp, preferably 100 bp or more, and more preferably 500 bp or more.

The “gene” of the present invention includes that constituted of DNA or RNA.

Further, in the present invention, the “nucleic acid” includes DNA and RNA.

Introduction of mutation to a gene can be generally effected by employing the conventional method such as Kunkel method and Gapped duplex method or a method equivalent thereto. For example, introduction of mutation to a gene is effected by using a kit for introducing mutation (for example, Mutant-K or Mutant-G manufactured by TAKARA Co., Ltd.) which utilizes site-specific mutagenesis method, or using the “LA PCR in vitro Mutagenesis” series kit, manufactured by TAKARA Co., Ltd.

Furthermore, it is acceptable that the amino acid sequence of the carrot-derived PSK receptor obtained as described above is used for database search and thereby a sequence which is homologous with the carrot-derived PSK receptor gene sequence is identified among the EST sequences of plants of various types. A gene which is homologous with the carrot-derived PSK receptor gene (i.e., a homologue) can be isolated by using the homologous sequence as a probe. Alternatively, such a homologue can easily be isolated, for example, by designing degenerate primers on the basis of the known amino acid sequence of the carrot-derived PSK receptor, preparing a template cDNA library from the target plant, and carrying out degenerate PCR by using the degenerate primers and the template cDNA library. In consideration of the homology of the deduced amino acid sequence of the isolated homologue, it is assumed that the homologue-encoding protein also has a similar function to that of the carrot-derived PSK receptor.

### 2. Preparation of Recombinant Vector Containing the Gene of the Present Invention

The recombinant vector of the present invention can be obtained by inserting the gene of the present invention to an appropriate vector. The vector to which the gene of the present invention is inserted is not particularly limited, as long as the vector enables replication in a host. Examples thereof include plasmid DNA, phage DNA and the like. Specific examples of plasmid DNA include a plasmid for Escherichia coli as a host such as pBR322, pBR325, pUC118, pUC119; a plasmid for Bacillus subtilis such as pUB110, pTP5; a plasmid for yeast as a host such as YEp13, YEp24 and YCp50; and a plasmid for a plant cell as a host such as pBI221 and pBI121. Specific examples of phage DNA include λ phage and the like. Alternatively, animal virus such as retrovirus and vaccinia virus, insect virus vector such as baculovirus, and plant virus may be used as a vector. When the gene of the present invention is inserted into a vector, there is employed a method including, for example, the steps of: cleaving purified DNA by treatment with an appropriate restriction enzyme; inserting the gene of the present invention into a restriction site or a multi-cloning site of an appropriate vector DNA; and connecting the gene to the vector. It is necessary that the gene of the present invention is incorporated to the vector such that the function of the gene can be fully effected. Therefore, the vector of the present invention may optionally contain cis element such as an enhancer, a splicing signal, a poly(A)-addition signal, a selective marker, ribosome binding sequence (SD sequence), or the like, as well as a promoter and the gene of the present invention. Examples of the selective marker include the dihydrofolate reductase gene, the ampicillin-resistant gene and the neomycin-resistant gene.

Specifically, the recombinant vector of the present invention can be prepared by inserting the carrot-derived PSK receptor gene of the present invention to binary vector pBI121, in sense or antisense orientation, under the control of the constitutive cauliflower mosaic virus 35S promoter incorporated within the binary vector.

### 3. Production of Transformant (Transgenic Plant) to Which the Gene of the Present Invention has Been Introduced

In the present invention, a transformant in which the PSK receptor protein-encoding gene has been introduced is also referred to as “a sense transformant”, and a transformant in which the antisense gene has been introduced is also referred to as “an antisense transformant”.

The portion of a plant, as the object of the transformation in the present invention, may be any of the following: a plant as a whole; organs of the plant (such as leaf, petal, stem, root and seed); plant tissues (such as epidermis, phloem, parenchyma, xylem and vascular bundle); and cultured cells of the plant. Plants of any type may generally be used for transformation. Monocotyledons such as rice, corn, asparagus and wheat and dicotyledons such as Arabidopsis thaliana, tobacco, carrot, soybean, tomato and potato, are especially preferable.

Any appropriate conventional method known in the art may be employed as a method of producing the transformant of the present invention. For example, the aforementioned recombinant vector may be introduced to a plant by the conventional transformation method such as electroporation method, Agrobacterium method, particle gun method, PEG method or the like.

In a case in which Agrobacterium method is employed, the recombinant vector of the present invention is introduced to an appropriate Agrobacterium such as Agrobacterium tumefaciens, and an axenic-cultured leaf piece of a host is infected with the resultant Agrobacterium strain according to vacuum infiltration method (Bechtold et al. (1993) C. R. Acad. Sci. Ser. III Sci. Vie, 316, 1194-1199), whereby a transgenic plant can be obtained.

In a case in which particle gun method is employed, the method may directly be applied to the plant as a whole, the plant organ or the plant tissue. Alternatively, the method may be applied after a section of the plant tissue is prepared. Or, the method may be applied after a protoplast is prepared. The samples prepared as describe above can be treated with a gene introducing device (e.g., BIOLISTIC POS 1000/He and BioRad). The treatment is generally conducted at a pressure of about 1000 to 1100 psi and a distance of 5 to 10 cm or so, although the treatment condition may vary depending on the type of the sample and the type of the plant.

The tumor tissue, shoot, hairy root and the like obtained as a result of transformation can directly be used for cell culture, tissue culture or organ culture. Further, the cultured cell obtained as a result of transformation can be regenerated to a plant, by administering plant hormones (such as auxin, cytokinin, gibberellin, abscisic acid, ethylene and brassinolide) at appropriate concentrations, according to the conventional plant tissue culture method.

In a case in which the transformant is a plant cell or a plant tissue, the regeneration of a plant can be conducted by using a conventional culture medium for plant culture, such as MS basal medium (Murashige, T. & Skoog, F. (1962) Physiol. Plant. 15: 473), LS basal medium (Linsmaier, E. M. & Skoog, F. (1965) Physiol. Plant. 18: 100) and the protoplast culture medium (which is a modified LS basal medium). With regard to the culture method, either the conventional solid culture method or liquid culture method can be employed. Culture is effected by inoculating 0.1 to 10 g fresh weight/L of cells, tissue or organ on the aforementioned medium and optionally adding NAA, 2,4-D, BA, kinetin or the like. The pH of the medium when the culture is started is adjusted in a range of 5.0 to 6.0, and the culture is conducted generally in a temperature range of 20 to 30° C. (preferably at 25° C. or so) with 10 to 120 rpm stirring for 2 to 4 weeks. In a case in which the transformant is a plant, the plant is grown by cultivation or hydroponic culture in a field or a glass house.

Specifically, for example, according to the protocol disclosed in M. Hardegger, A. Aturm, Mol. Breed. 4, 119 (1998), a transformant of the present invention can be obtained by introducing the aforementioned binary vector pBI121, having the carrot-derived PSK receptor gene incorporated thereto, to a host plant cell and regenerating to an entire plant.

Further, a transformant of the present invention can be obtained by introducing the receptor gene of the present invention not only to the aforementioned plant host, but also to a host including bacteria such as Escherichia coli, yeast, animal cells or insect cells, without being restricted to such examples. When bacteria such as Escherichia coli and yeast is used as a host, the recombinant vector of the present invention preferably contains a sequence enabling autonomous replication in the bacteria, a promoter, ribosome binding sequence, the gene of the present invention and the transcription termination sequence. The recombinant vector may further include a sequence which regulates the promoter.

Whether the PSK receptor gene of the present invention has been incorporated to the host or not can be confirmed by PCR method, Southern hybridization method, Northern hybridization method or the like. For example, in the PCR method, DNA is extracted as a template for PCR from the transformant, primers specific to the PSK receptor gene are designed, and PCR is carried out. PCR can be carried out in the substantially the same condition as in the preparation of the aforementioned plasmid. Thereafter, the PCR product obtained as a result of the amplification is subjected to agarose gel electrophoresis, polyacrylamide gel electrophoresis or capillary electrophoresis, and dyed by treatment with ethidium bromide, SYBR Green or the like. The amplified product is detected as a single band, and thereby it can be confirmed that the transformation is successful. Alternatively, a primer labeled in advance with fluorescence dye or the like may be used in PCR, so that the amplified product can be detected from fluorescence. Or, the amplified product may be bound to the solid phase of a microplate or the like, so that the amplified product can be detected from fluorescence or enzymatic reactions.

### 4. Purification of the Protein of the Present Invention

In a case in which the protein of the present invention is produced inside the transformed bacteria or cells, the target protein is collected by destroying the bacteria or cells by ultrasonic treatment, repeated freezing and melting, homogenizer treatment or the like. In a case in which the protein of the present invention is secreted outside the bacteria or cells, the target protein is collected directly from the culture medium or collected from the culture medium after removing the bacteria or cells therefrom with centrifugation. Thereafter, the protein of the present invention can be isolated and purified from the culture medium by employing the conventional biochemical methods for isolation and purification of proteins, such as ammonium sulfate precipitation, gel chromatography, ion exchange chromatography, affinity chromatography or the like. Each of these methods may be used singly. Alternatively, some of these methods may be employed in combination.

When the PSK receptor protein is to be purified from cultured cells or cultured tissue, cells are first destroyed by cell-lysis treatment with enzymes such as cellulase, pectinase or the like, ultrasonic treatment, milling or the like. Next, the insoluble components are removed by filtration or centrifugation, whereby a coarse protein solution is obtained. The PSK receptor protein of the present invention can be purified from the coarse protein solution by salting out, chromatography of various types (e.g., gel filtration chromatography, ion exchange chromatography, affinity chromatography) or SDS polyacrylamide gel electrophoresis, or combination thereof.

### 5. Production of Plant Whose Cell Division and Proliferation Have Been Enhanced, According to a Method of the Present Invention

A plant whose cell division and proliferation have been enhanced can be obtained by redifferentiating the transformed plant cell of the present invention. Specifically, the PSK receptor-encoding gene (i.e., sense gene) of the present invention is isolated as described above (and preferably incorporated it into a vector), the isolated gene is introduced to plant cells as described above, and the transformed plant cells thus obtained are cultured as described above. That is, the transformed plant cells are cultured in a growth medium where the cells are allowed to proliferate, and then in a redifferentiation medium where the cells are allowed to redifferentiate. Thereby, a regenerated plant is obtained. Thus obtained plant has an enhanced responsive property to PSK and therefore exhibits accelerated cell division and proliferation.

Similarly, a plant whose cell division and proliferation have been decreased can be obtained by redifferentiating the transformed plant cell which has been transformed with the antisense gene of the present invention.

### 6. Effect of the Present Invention

By overexpressing the PSK receptor according to the present invention, the growth rate of plant cells can be increased. This effect is semi-permanently maintained by the stimulation of PSK produced by the cells themselves. For example, when plant cells are made to overexpress the PSK receptor, the growth rate of the plant cells is enhanced. Conversely, when the plant cells are made to express the PSK receptor antisense mRNA, the growth rate of the plant cells can be decreased.

### EXAMPLE

### Methods

Preparation of PSK-based Affinity Column

For the preparation of [Lys⁵]PSK-Sepharose containing 6-aminohexanoic acid (Ahx) spacer, 210 mg (0.2 mmol) of Fmoc-Tyr(SO₃H)-Ile-Tyr(SO₃H)-Thr-Lys prepared by solid-phase synthesis was reacted with 1.0 mmol of Boc-(Ahx)₂-OSu in 5 ml of 50% acetonitrile in the presence of 1.0 mmol of NaHCO₃ at room temperature for 1.0 h (Y. Matsubayashi, H. Hanani, O. Hara, Y. Sakagami, Biochem. Biophys. Res. Commun. 225, 209 (1996)). The peptide containing a Boc-Ahx₂ tail was purified by reverse-phase HPLC, lyophilized, and treated with 6.0 ml of 95% trifluoroacetic acid at room temperature for 12 min to deprotect the Boc group. Deprotected peptide was immediately purified by reverse-phase HPLC, followed by lyophilization to afford Fmoc-Tyr(SO₃H)-Ile-Tyr(SO₃H)-Thr-Lys(^{ε}N-(Ahx)₂); yield, 180 mg (0.14 mmol, 70%). A 129-mg (0.1 mmol) sample of this peptide was dissolved in 10 ml of 50% acetonitrile containing 2.0 mmol of NaHCO₃ and coupled to 5.0 ml of prepacked Hi-Trap NHS activated Sepharose (Amersham Pharmacia Biotech) according to the manufacturer's protocol. After deactivation of the unreacted NHS groups by 0.2 M ethanolamine, the ligand-coupled Sepharose was treated with piperidine:acetonitrile:water (2:1:1) for 10 min to deprotect the Fmoc groups. Coupling efficiency was 10.8 μmol ligand/5.0 ml Sepharose, as determined by measuring absorbance of released fluorescence derivative at 301 nm. The column was thoroughly washed with dimethylformamide, 50% acetonitrile and water before use. Because Hi-Trap NHS activated Sepharose contains Ahx linker between Sepharose and NHS groups, this affinity matrix contains triple Ahx spacer between [Lys⁵]PSK moiety and Sepharose.

Affinity Purification of PSK-Binding Proteins

Carrot microsomal membranes (1,200 mg protein) were solubilized in 320 ml of buffer containing 20 mM HEPES-KOH (pH 7.5), 50 mM KCl, and 1.0% Triton X-100 (buffer A). Solubilized materials were centrifuged at 100,000 g for 30 min at 4° C., and supernatants were applied to the [Lys⁵]PSK-Sepharose column (5.0 ml) at a flow rate of 0.5 ml/min using the AKTA prime chromatography system (Amersham Pharmacia Biotech). After washing with 50 ml of buffer containing 20 mM HEPES-KOH (pH 7.5), 50 mM KCl, and 0.1% Triton X-100 (buffer B), the column was eluted with 15 ml of 1.0 mg/ml PSK in buffer B. The eluates were added to a 1.0-ml column of Macro-Prep Ceramic Hydroxyapatite Type I (Bio-Rad laboratories) at a flow rate of 0.5 ml/min at 4° C. The column was washed with 20 ml of buffer B and eluted with a 18-ml gradient of KH₂PO₄ (0 to 400 mM) in buffer B. Active fractions (12 ml), as determined by [³H]PSK binding assay, were concentrated by ultrafiltration (Ultrafree-15 with Biomax-10 membranes, Millipore) and analyzed by SDS-PAGE using 7.5% gels (Y. Matsubayashi, Y. Sakagami, Eur. J. Biochem. 262, 666 (1999)).

Tryptic Digestion of 120-kD Protein

For large-scale purification and tryptic digestion of 120-kD protein, affinity-purified proteins were precipitated by acetone, reduced by dithiothreitol, and pyridylethylated prior to electrophoresis (U. Hellman, C. Wernstedt, J. Gonez, C. H. Heldin, Anal. Biochem. 224, 451 (1995)). After SDS-PAGE and Nile red staining, each band was excised and subjected to in situ digestion with TPCK-trypsin (Sigma) (U. Hellman, C. Wernstedt, J. Gonez, C. H. Heldin, Anal. Biochem. 224, 451 (1995)). Resultant peptides were extracted from the gel, concentrated in vacuo, and separated on a TSKgel ODS-80TS (2.0×150 mm, Tosoh, Japan) by 140-min gradient elution with 10 to 50% acetonitrile in 0.1% trifluoroacetic acid at a flow rate of 100 μl/min using a 140A solvent delivery system (Applied Biosystems).

Nested PCR Using Degenerated Primers

Based on the sequences of e and f, degenerated primers E1 (5′-GGYTCYTCNACNGCRTTYTC-3′ (SEQ ID No: 3)), E2 (5′-TTRAARAANGGRAARTCNGG-3′ (SEQ ID No: 4)), and F (5′-GTNTAYGARAAYTCNTTYCA-3′ (SEQ ID No: 5)) were synthesized. The first PCR was performed with primers E2 and F, using the first-strand cDNA prepared from NC cells as a template. The temperature was set at 95° C. for 60 seconds, 45° C. for 60 seconds and 72° C. for 120 seconds, with the amplification cycle being repeated 40 times. The PCR products were used as templates for nested PCR, using the second primers E1 and F. PCR products were subcloned and used for isolation of the cDNA.

Isolation of Full Length cDNA of PSK Receptor-Encoding Gene

The aforementioned PCR product which had been subcloned in a pBS SK vector was cleaved with EcoRV, and a marker probe was prepared from the cleaved DNA fragment by using AlkPhos Direct Kit manufactured by Amersham Pharmacia Co., Ltd. 100,000 plaques of the carrot NC cell-derived cDNA library phage, which has been prepared by using ZAP-cDNA Synthesis Kit (manufactured by STRATAGENE Co., Ltd.), were grown on a LB culture medium, and these plaques were transferred and fixed on a nylon membrane. The membrane was subjected to hybridization with the marker probe by using a reagent attached to the AlkPhos Direct Kit, according to the protocol thereof. Thereafter, the positive plaques were detected by the Detection Kit manufactured by Amersham Pharmacia Co., Ltd. The inserted portion (i.e., full length cDNA) in the positive phage was subcloned to pBS vector, by using the helper phage attached to the Kit. The full length cDNA sequence was analyzed with the 310-type sequence analyzer manufactured by Applied Biosystems Co., Ltd.

Genetic Transformation of Carrot Cells

The chimeric genes were composed of the receptor kinase ORF, in the sense or antisense orientation, under the control of the constitutive cauliflower mosaic virus 35S promoter incorporated within the binary vector pBI 121. Transformation of carrot hypocotyl segments and plant regeneration were performed following a protocol described elsewhere (M. Hardegger, A. Sturm, Mol. Breed. 4, 119 (1998)). Carrot cells transformed with the binary vector alone were used as controls. Analysis of variance of the growth data was carried out using the Student's t-test procedure of the Prism software (GraphPad Software).

Immunoprecipitation of the Photoaffinity Labeled Proteins

An extracellular domain of the 120-kD receptor kinase (excluding the signal peptide) was expressed in E. coli using pET-24b expression vector (Novagen) and purified as a His₆ fusion. This recombinant protein was used as an antigen for generating the antibodies in rabbits (MBL, Nagoya, Japan), and for affinity purification of the antibodies using Hi-Trap NHS activated Sepharose (Amersham Pharmacia Biotech). Western blotting was performed using ECL (Amersham Pharmacia Biotech) according to the manufacturer's protocol. For the immunoprecipitation of the photoaffinity labeled proteins, labeled membrane proteins (50 μg) were solubilized with 50 μl of buffer A, and immunoprecipitated using purified antibodies or IgG fraction of pre-immune as a control and rProtein A Sepharose (Amersham Pharmacia Biotech). The Sepharose beads were boiled in electrophoresis sample buffer and the supernatant was analyzed by SDS-PAGE.

### Result and Discussion

Photoaffinity Labeling and PNGase Treatment of the PSK-Binding Proteins

Photoaffinity labeling of carrot cell line NC membrane proteins with a photoactivatable PSK analog and the subsequent SDS-PAGE analysis indicated that an approximately 120-kD protein and an approximately 150-kD protein specifically interact with PSK (FIG. 1). Also, it was revealed that both of these PSK-binding proteins contain N-linked oligosaccaride chains of approximately 10 kD that can be cleaved by treatment with peptide N-glycosidase F (PNGase F).

SDS-PAGE Analysis of Affinity-Purified Proteins

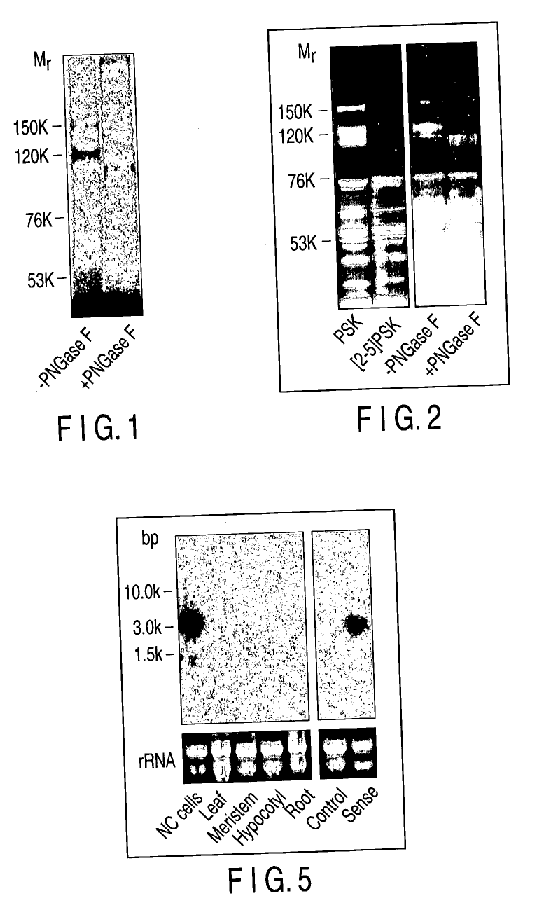

The PSK-binding proteins were purified with [Lys⁵] PSK-Sepharose column. The purified proteins were further purified by hydroxyapatite column chromatography, concentrated and subjected to SDS-PAGE and Nile red staining. The results indicate that a major protein of approximately 120-kD and a minor protein of approximately 150-kD are specifically recovered (FIG. 2). Both of these proteins were absent in the fractions eluted by [2-5]PSK, which is a synthetic analog of PSK, and exhibited no biological activity or binding activity (FIG. 2). PNGase F treatment of these two proteins decreased the apparent sizes thereof to 110 kD and 140 kD, respectively, suggesting that the two proteins are identical to the proteins detected in the photoaffinity cross-linking experiments (FIG. 2; see also FIG. 1).

Reversed-Phase HPLC Profile of the Tryptic Digest of the Purified 120 kD Protein

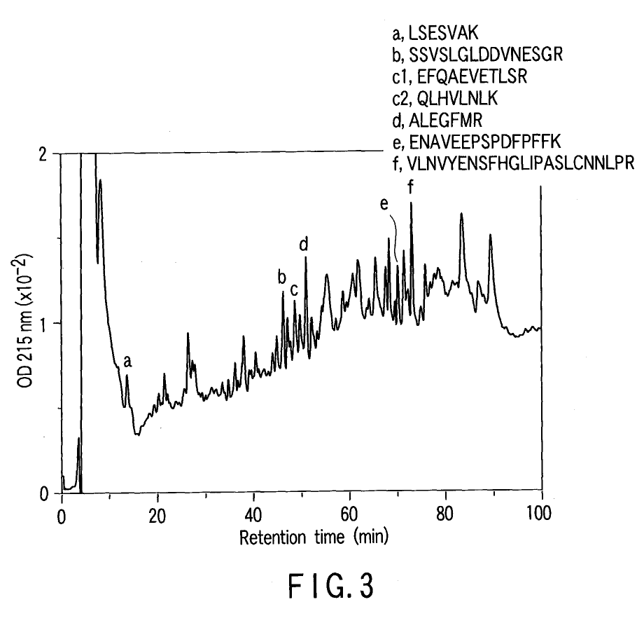

Four independent purifications were performed, yielding 50 μg of the major 120-kD protein from 4800 mg of microsomal proteins, with an overall recovery rate of 40%. The protein was digested with TPCK-trypsin (TPCK, tosyl phenylalanyl chloromethyl ketone), and peptide fragments thus generated were separated by reversed-phase high-performance liquid chromatography (HLPC) (FIG. 3). The fragments of the 120-kD protein contained in 15 independent peaks were analyzed, using a protein sequencer and MALDI-TOF MS (matrix-assisted laser desorption/ionization time-of-flight mass spectrometry), whereby the complete amino acid sequences of seven internal peptides were obtained from six peaks (FIG. 3, peaks a to f).

Cloning of the 120-kD Protein

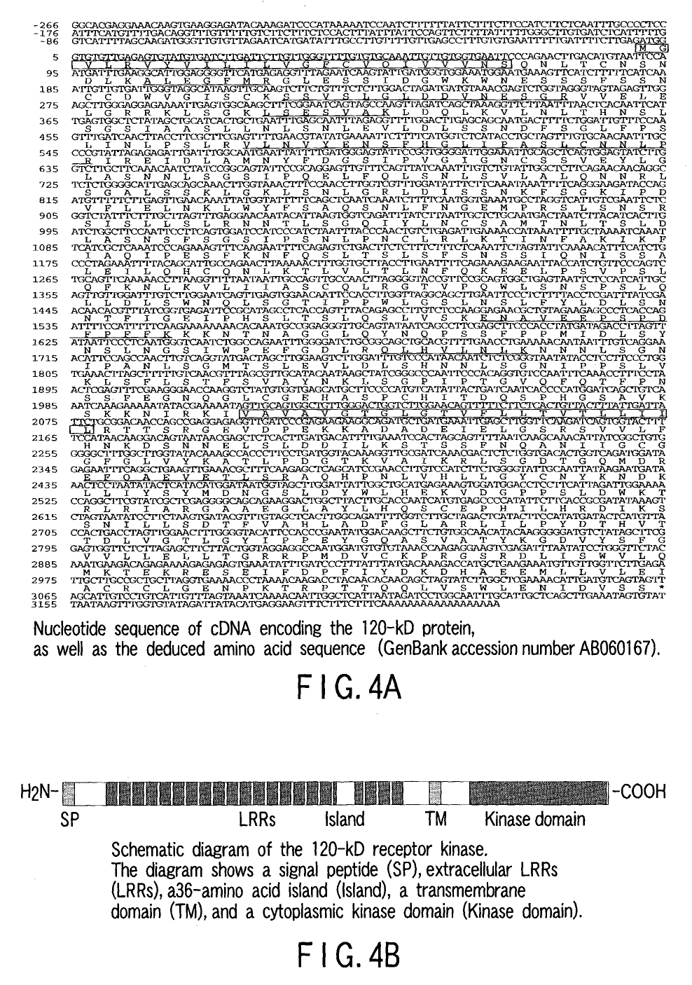

Of the seven internal peptides of the 120-kD protein, amino acid sequences of three peptides (cl, e, and f) were used to synthesize degenerate oligonucleotides, which were used as nested primers in PCR amplification of first-strand cDNAs of carrot NC cell. Of the six primer pairs tested, a specific PCR product was obtained only with the primer set based on peptides e and f. Using the PCR product as a hybridization probe, the cDNA library of carrot NC cell was screened, and a 3.5-kb cDNA clone was isolated. Analysis of the amino acid sequence of the longest open reading frame revealed that the cDNA encoded a 1021-amino acid protein, with a deduced molecular mass of 112 kD (FIG. 4A). It was also revealed that this protein contained an NH₂-terminal hydrophobic signal sequence, extracellular leucine-rich repeats (LRRs), a transmembrane domain, and a cytoplasmic kinase domain (FIG. 4B).

Northern Blot Analysis of the 120-kD Receptor Protein mRNA

Northern blot analysis was conducted in order to examine the expression pattern of the corresponding gene (i.e., 120-kD receptor protein-encoding gene) (FIG. 5). The total RNA was isolated from the NC cells, various parts of 2-week-old carrot seedlings, and the transformed cells, for analysis, respectively. The mRNA accumulated ubiquitously in leaf, apical meristem, hypocotyl, and root of carrot seedlings, although the expression level in the carrot seedlings was lower than that in cultured NC cells. In FIG. 5, “rRNA” represents ribosome RNA and “bp” represents the number of base pairs.

Overexpression of the Receptor Protein

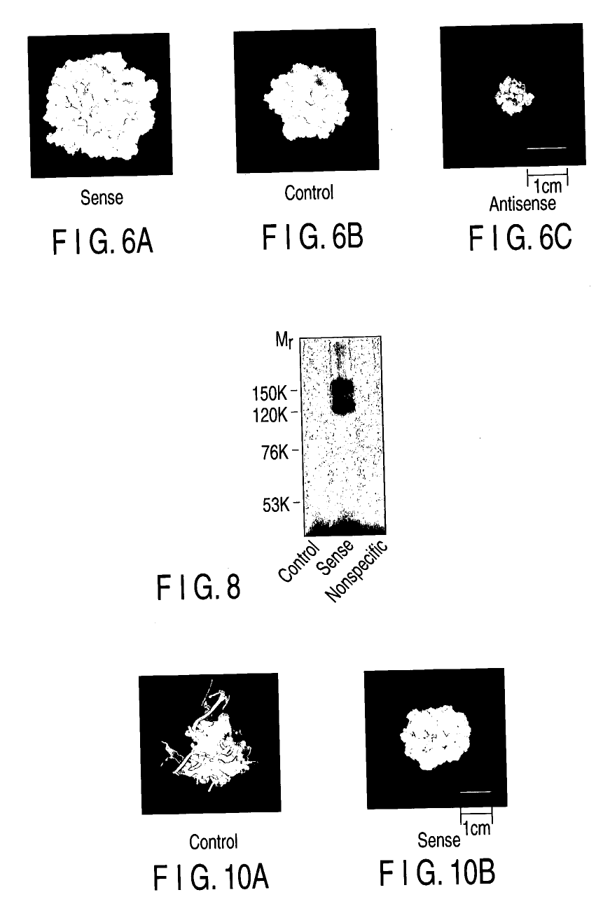

The cDNA of the aforementioned protein was overexpressed in transgenic carrot cells, in sense orientation, under the control of the cauliflower mosaic virus 35S promoter. This transgenic carrot cells exhibited accelerated growth in response to PSK, as compared with control cells (FIG. 6A and FIG. 6B). In contract, expression of the antisense strand substantially inhibited callus growth of transgenic carrot cells that is transformed with an antisense gene (FIG. 6C). These phenotypes are consistent with the hypothesis that overexpression and antisense inhibition of this receptor protein alter the responsive property of carrot cells to PSK. FIGS. 6A to 6C show the callus growth of the sense transformants, the control cells, and antisense transformants exposed to 10 nM PSK, respectively. The transformed carrot cells and the control cells were cultured for 3 weeks on B5 media containing naphthaleneacetic acid (NAA, 1.0 mg/liter), 6-benzylamino purine (6-BA, 0.5 mg/liter), and 10 nM PSK. Representative data of one of three independent experiments are shown in FIGS. 6A to 6C. The scale bar in FIG. 6C represents 1 cm.

Increase in PSK Binding Activity

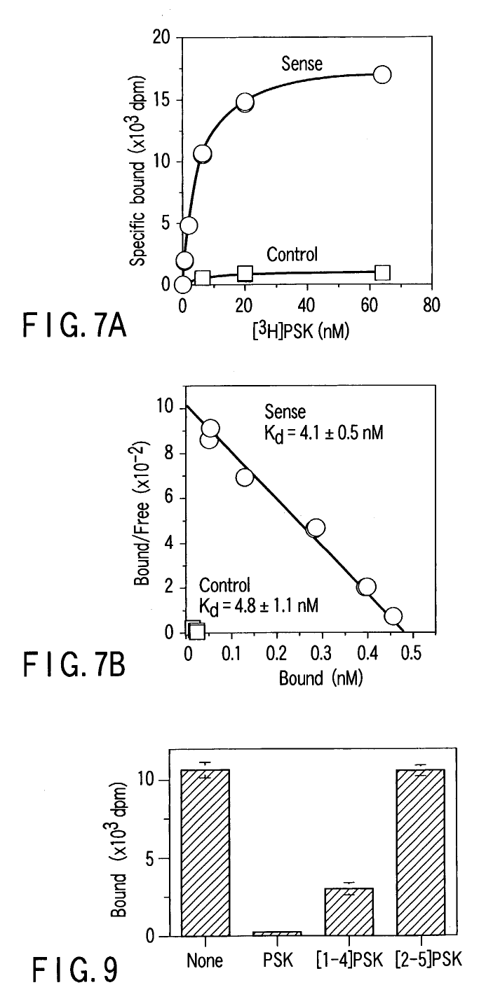

FIG. 7A is a graph which shows specific binding of PSK to the receptor protein in the sense transformants and the control cells, respectively. FIG. 8 is a photograph which shows the result of photoaffinity labeling of the membrane proteins derived from the control cells and the sense transformants. A sizable increase in PSK binding activity in the membrane fractions of the sense transformants is observed (FIG. 7A and FIG. 8). FIG. 7B is a graph which shows Scatchard plot of the binding data in FIG. 7A. It is understood, from the result of FIG. 7B, that the increase in PSK binding was due to an increase in the number of binding sites [sense transformant, Bₘₐₓ=570±18 fmol per mg of membrane protein; control Bₘₐₓ=34±2 fmol per mg of membrane protein (Bₘₐₓ being the maximum number of binding sites)], with similar binding affinities (sense transformant, Kd=4.1±0.5 nM; control, Kd=4.8±1.1 nM). The photoaffinity labeling analysis and immunoprecipitation analysis of the membrane protein derived from the sense transformants revealed that both the 150-kD protein and the 120-kD protein are encoded by a single gene.

Specificity of the PSK Binding Activity

The specificity of the PSK binding activity was characterized by comparing the relative binding affinity for several PSK analogs. FIG. 9 is a graph which shows the relative binding affinity when the binding of [³H]PSK to the membrane fraction of the sense transformant was inhibited by the competitor PSK, [1-4]PSK or [2-5]PSK that is unlabelled (In FIG. 9, the error bars indicate ± SE (standard error) from three independent experiments). The membrane proteins were incubated in binding buffer containing 6.3 nM [³H]PSK and 3.2 μM of the competitor . The binding of [³H]PSK to the membrane fraction of the sense transformant was strongly inhibited by unlabeled PSK, less strongly inhibited by the less active analog [1-4]PSK, and not inhibited at all by the inactive analog [2-5]PSK. Such high specificity and affinity for PSK strongly suggest that the aforementioned receptor protein directly interacts with PSK and thus is a component of a functional PSK receptor.

Proliferation of the Transformants

FIG. 10A is a photograph showing control cells and FIG. 10B is a photograph showing transformed cells which express high levels of sense mRNA of the aforementioned receptor. The scale bar in FIG. 10B represents 1 cm. Transformed carrot cells and control cells were cultured for 4 weeks on B5 media without plant hormones, to induce plant regeneration. The transformed cells exhibited accelerated proliferation, but were not able to regenerate roots and shoots.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A phytosulfokine (PSK) receptor protein selected from the groups consisting of:
(a) a protein comprising an amino acid sequence of SEQ ID No: 2; and
(b) a protein comprising an amino acid sequence of SEQ ID No: 2, wherein one or a few amino acids are deleted, substituted and/or added and which is capable of responding to phytosulfokine (PSK) that is a plant cell growth factor.

2. A gene encoding a phytosulfokine (PSK) receptor protein, said protein being selected from the groups consisting of:
(a) a protein comprising an amino acid sequence of SEQ ID No: 2; and
(b) a protein comprising an amino acid sequence of SEQ ID No: 2, wherein one or a few amino acids are deleted, substituted and/or added and which is capable of responding to phytosulfokine (PSK) that is a plant cell growth factor.

3. A gene encoding a phytosulfokine (PSK) receptor protein, said gene being selected from the groups consisting of:
(c) a gene having a nucleotide sequence of SEQ ID No: 1;
(d) a gene having a nucleotide sequence of SEQ ID No: 1, wherein one or a few nucleotides are deleted, substituted and/or added and which encodes a protein that is capable of responding to phytosulfokine (PSK) that is a plant cell growth factor; and
(e) a gene having a nucleotide sequence which can hybridize with a complementary strand of a nucleotide sequence of SEQ ID No: 1 under a stringent condition and which encodes a protein that is capable of responding to phytosulfokine (PSK) that is a plant cell growth factor.

4. An antisense gene having a nucleotide sequence complementary to the gene according to claim 2.

5. An antisense gene having a nucleotide sequence complementary to the gene according to claim 3.

6. A recombinant vector containing the gene according to claim 2.

7. A recombinant vector containing the gene according to claim 3.

8. A recombinant vector containing the antisense gene according to claim 4.

9. A recombinant vector containing the antisense gene according to claim 5.

10. A transformant having the gene according to claim 2.

11. A transformant having the gene according to claim 3.

12. A transformant having the antisense gene according to claim 4.

13. A transformant having the antisense gene according to claim 5.

14. A transgenic plant having the gene according to claim 2.

15. A transgenic plant having the gene according to claim 3.

16. A transgenic plant having the antisense gene according to claim 4.

17. A transgenic plant having the antisense gene according to claim 5.

18. A method of preparing a plant cell whose responsive property to a plant cell growth factor PSK is enhanced, comprising:
introducing the gene according to claim 2 into a plant cell, thereby obtaining the transformed cell; and
culturing the transformed plant cell in a medium where the cell is allowed to proliferate.

19. A method of preparing a plant cell whose responsive property to a plant cell growth factor PSK is enhanced, comprising:
introducing the gene according to claim 3 into a plant cell, thereby obtaining the transformed cell; and
culturing the transformed plant cell in a medium where the cell is allowed to proliferate.

20. A method of preparing a redifferentiated plant whose responsive property to a plant cell growth factor PSK is enhanced, comprising:
introducing the gene according to claim 2 into a plant cell, thereby obtaining the transformed cell;
culturing the transformed plant cell in a growth medium where the cell is allowed to proliferate; and
redifferentiating the proliferated cell in a redifferentiation medium where the cell is allowed to redifferentiate.

21. A method of preparing a redifferentiated plant whose responsive property to a plant cell growth factor PSK is enhanced, comprising:
introducing the gene according to claim 3 into a plant cell, thereby obtaining the transformed cell;
culturing the transformed plant cell in a growth medium where the cell is allowed to proliferate; and
redifferentiating the proliferated cell in a redifferentiation medium where the cell is allowed to redifferentiate.
